(19) United States
(12) Patent Application Publication
(10) Pub. No.: US 2009/0299349 A1
(43) Pub. Date: Dec. 3, 2009

(54) LIGHT IRRADIATING DEVICE

(75) Inventors: Hiroyuki Kubota, Kanagawa (JP); Takeo Ishii, Kanagawa (JP); Yukinori Kubotera, Kanagawa (JP)

Correspondence Address: BUCHANAN, INGERSOLL & ROONEY PC, POST OFFICE BOX 1404, ALEXANDRIA, VA, 22313-1404, US

(73) Assignee: TERUMO KABUSHIKI KAISHA, Tokyo (JP)

(21) Appl. No.: 12/090,457

(22) PCT Filed: Oct. 17, 2006
(86) PCT No.: PCT/JP2006/320602

§ 371 Date: Jun. 27, 2008

(30) Foreign Application Priority Data
Oct. 18, 2005 (JP) 2005-303567

(51) Int. Cl. A61B 18/20

(52) U.S. Cl. 606/9

(57) ABSTRACT

A light irradiating device capable of irradiating with a low output energy a lesion or a skin deep portion with light having a high-vasodilating-effect wavelength. Light including a wavelength having a vasodilating effect is emitted from the output portion at the tip end of a probe. The output density of the light is preferably 100 to 1550 mW/mm². The wavelength of light having a vasodilating effect is preferably 450 to 650 nm. A skin contact surface is preferably formed at the tip end of the probe, and the output portion is preferably disposed at the skin contact surface.

### TECHNICAL FIELD

The present invention relates to a light irradiating device for irradiating a skin with light, for example, a light irradiating device for irradiating with a low output a skin with light including a wavelength having a vasodilating effect.

### BACKGROUND ART

In recent years, in pain clinic and dermatological fields, phototherapeutic apparatuses such as low reaction level laser (low-output laser) therapeutic apparatus and linearly polarized infrared ray therapeutic apparatus have widely been used for treatment of pains in the vicinity of the skin surface, such as postoperative or posttraumatic wound pain, posttraumatic pain, zoster pain, postzoster neuralgia, etc. and skin diseases such as dermal ulcer, diabetic circulatory incompetency, Raynaud's disease, Buerger's disease, alopecia greata, etc.

The low reaction level laser therapeutic apparatuses have an output of 60 to 1000 mW in the case of general-purpose products, and normally have a single laser output portion. In these apparatuses, the laser beam diameter at the output portion is 1.4 to 13.8 mm, and the output density is 680 to 9600 mW/cm². In addition, the laser beam diameter increases gradually as the beam goes away from the output portion. On the other hand, the linearly polarized near infrared ray therapeutic apparatuses have an output of 500 to 2200 mW, and ordinarily have a single infrared ray output portion.

As a common action mechanism involved in the amelioration of pain and the amelioration of circulatory disorder during treatment of skin diseases by use of these phototherapy apparatuses, the vasodilating effect of light has come to draw attention. For example, dilation of dermal blood vessels causes diffusion and removal of pain-related substances (bradykinin, histamine, prostaglandin, etc.) from the local site, whereby the pain is alleviated and the skin can be sufficiently fed with oxygen and nutrition.

Besides, a direct relaxing effect on the vascular smooth muscles has come to be known as a principal mechanism of the circulation-ameliorating effect. Recently, it has been elucidated that production of nitrogen monoxide (NO) is relating to the relaxation of smooth muscles by light.

However, although the above-mentioned phototherapy apparatuses in the related art have been highly evaluated in view of few side effects, it has come to be pointed out that these apparatuses have drawbacks of their insufficient therapeutic effects and the long periods of time needed for therapy.

Patent Document 1 reports that light on the shorter wavelength side is effective in producing an enhanced phototherapeutic effect. In fact, while the wavelength of light used in the phototherapy apparatuses is 810 to 830 nm in the case of laser and is 600 to 1600 nm (peak: 1000 nm) in the case of linearly polarized near infrared rays, it has been found out that the circulation ameliorating (e.g., vasodilating) effect as one of the mechanisms of the analgesic process is greater on the shorter wavelength side. According to Patent Document 1, particularly, light in the visible wavelength region (near 532 nm) has a strong relaxing effect on blood vessels.

Patent Document 1: Japanese Patent Laid-Open No. 2000-187157

### DISCLOSURE OF THE INVENTION

### Problems to be Solved by the Invention

Although a phototherapeutic apparatus using light in a shorter wavelength region as described in Patent Document 1 is expected to produce a sufficient effect, the apparatus has a drawback that the light with the shorter wavelength is poor at reaching the depth of the tissue. Therefore, in order that the light radiated from above the skin can sufficiently reach a lesion present at a skin deep portion, the output energy of the light for irradiation has to be comparatively high, and direct irradiation with the light at such a high output energy may injure a skin surface layer portion with a size of several millimeters to several tens of millimeters.

### Means for Solving the Problems

In order to solve the above-mentioned problems, according to one embodiment of the present invention, there is provided a light irradiating device for emitting light including a wavelength having a vasodilating effect, from an output portion provided at the tip end of a probe, wherein the output density of the light is 100 to 1550 mW/mm².

### EFFECT OF THE INVENTION

According to the light irradiating device based on the present invention, light with a predetermined wavelength can be made to reach a skin deep portion.

### BRIEF DESCRIPTION OF DRAWINGS

FIG. 1 is a front view showing schematically a light irradiating device according to a first embodiment of the present invention.

FIG. 2 is a bottom view of the light irradiating device shown in FIG. 1.

FIG. 3 is a schematic view of a light irradiating device according to a second embodiment of the present invention.

FIG. 4 is an illustration showing the results of measurement on a bloodstream measuring apparatus.

### BEST MODE FOR CARRYING OUT THE INVENTION

Now, the light irradiating device according to the present invention will be described in detail below, based on the drawings.

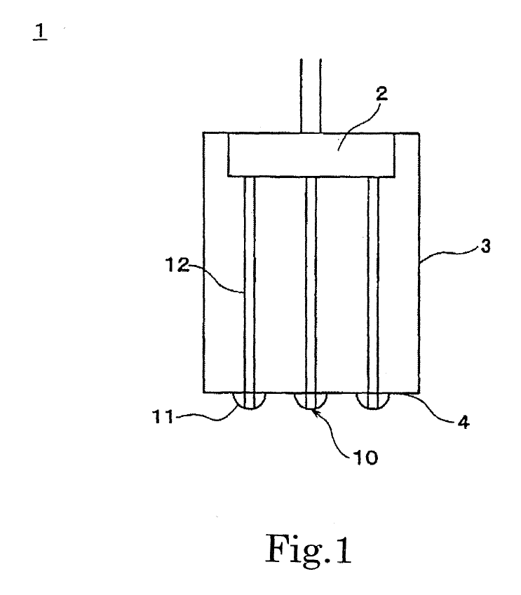

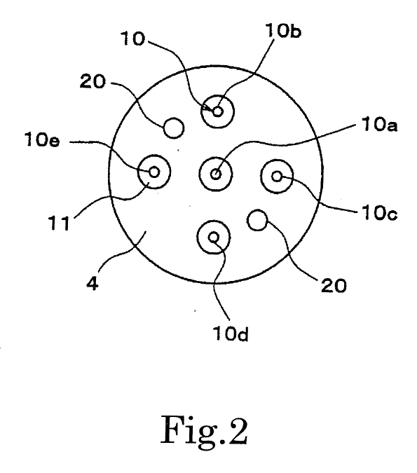

FIG. 1 is a front view showing schematically a light irradiating device according to a first embodiment of the present invention, and FIG. 2 is a bottom view of the same. As shown in FIGS. 1 and 2, the light irradiating device 1 according to the first embodiment is configured as a laser irradiating device for irradiating a skin with laser beams. The laser irradiating device 1 has a probe 3, which has a hollow cylindrical shape, with a laser device 2 disposed in an upper end portion of the probe 3. In addition, the probe 3 is provided at its lower portion with a flat and smooth skin contact surface 4.

Output portions 10 for emitting laser beams are disposed at the skin contact surface 4. Each of the output portions 10 is opened in a central area of a semispherical projected portion 11 rising from the skin contact surface 4. The height of the projected portions, which determines the distance from the skin contact surface 4 to the skin surface, is set to be 1 to 5 mm, for example.

While the number of the output portion 10 may be one, a plurality of output portions 10 are provided in this embodiment. Specifically, the output portions 10 are disposed at a central portion (10a) of the circular skin contact surface 4, and at four locations (10b, 10c, 10d, 10e) in the surroundings thereof which are spaced at regular intervals along the circumferential direction. In the case where a plurality of the output portions 10 are disposed at the skin contact surface 4 of the probe 3, the interval between the output portions is set in the range of 4 to 10 mm, preferably 6 to 8 mm. In addition, the output of each of the output portions 10 is set to 10 mW or below.

A fiber 12 extending from the laser device 2 is connected to each output portion 10, and a laser beam at a wavelength having a vasodilating effect is emitted from the output portion 10. The wavelength of the laser beam having the vasodilating effect is in the range of 450 to 650 nm. While the laser irradiation is sustained irradiation here, it may be pulsed irradiation.

The diameter of each of the fibers 12 is set in the range of 0.5 to 0.02 mm, preferably 0.2 to 0.05 mm. The minimum diameter of the fibers 12 is thus set to 0.02 mm, because fibers smaller in diameter are more difficult to manufacture. Normally, the lower limit of diameter in manufacturing plastic fibers is considered to be 0.1 mm, and that of glass fibers is considered to be 0.01 mm. On the other hand, the maximum diameter of the fibers 12 is set at 0.5 mm, because a fiber diameter in excess of 0.5 mm may cause a pain through a thermal action in the cases where the output density is high. On the other hand, where the fiber diameter is small (0.2 mm or below), it is considered that no or extremely little pain will be generated even upon a thermal stimulus action of not less than 4° C. For example, the 31G needle (outer diameter: 0.25 mm) for self-injection of insulin is known to produce little puncture pain.

The output density of the laser beam emitted from each output portion 10 is set in the range of 100 to 1550 mW/mm², as above-mentioned. As will be described later, an output density of less than 100 mW/mm² results in that the vasodilating effect is weak, and the effect is limited to the irradiated portion. On the other hand, an output density in excess of 1550 mW/mm² would cause a thermal action to come to the front, possibly producing a pain. With the output density thus set within the range of 100 to 1550 mW/mm², a positive vasodilating effect is exerted not only on the irradiated portion but also on the neighborhood (broadly in horizontal directions through axon reflex). For example, in the case where the fiber diameter is 0.2 mm and where irradiation with an output of 3 mW (which promises the vasodilating effect described later) is conducted with an irradiation area of 0.0314 mm², the output density is 95.54 mW/mm². On the other hand, in the case where the fiber diameter is 0.05 mm and where irradiation with an output of 3 mW is conducted with an irradiation area of 0.00196 mm², the output density is 1530 mW/mm².

A touch sensor 20 which operates upon being contacted by a skin is provided in the vicinity of the output portions 10, and the laser beams are emitted from the output portions 10 only when the touch sensor 20 is operating. In this embodiment, two touch sensors 20 are provided which are each located between adjacent ones of the output portions (between 10b and 10e, and between 10c and 10d) and which are arrayed with each other along a diametral direction of the skin contact surface 4 of the probe 3. A configuration is adopted in which, for example, the emission of the laser beams from the output portions 10 occurs only when both of the touch sensors 20 make contact with a skin.

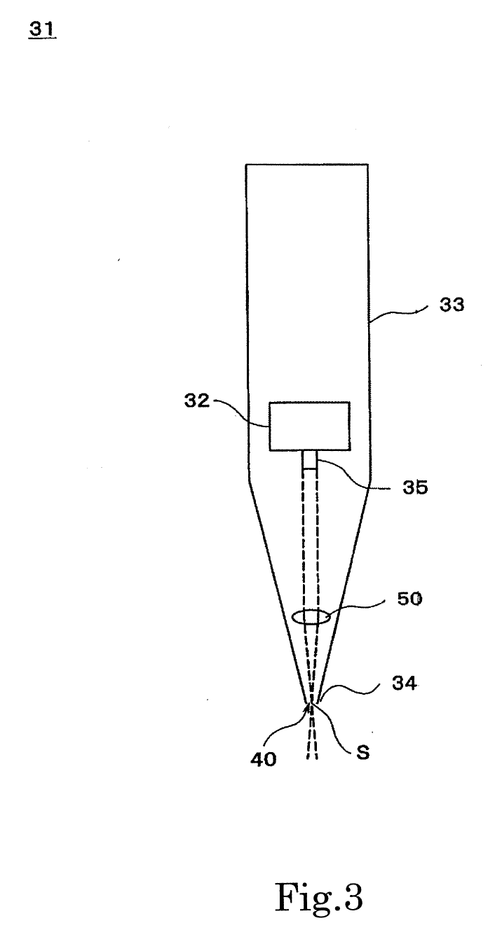

In the next place, FIG. 3 is a schematic view of a light irradiating device according to a second embodiment of the present invention. As shown in the figure, the light irradiating device 31 in the second embodiment is configured as a laser irradiating device for irradiating a skin with a laser beam, and is different from the first embodiment in type. A probe 33 of the laser irradiating device 31 has a cylindrical shape, with a lower half being gradually decreased in diameter.

A skin contact portion 34 is formed at the lower end portion of the probe 33, and an output portion 40 for emitting a laser beam is opened in the skin contact portion 34. A laser device 32 is contained in a central portion of the inside of the probe 33, and has a light emitting portion 35 fronting on the output portion 40. In addition, between the output portion 40 and the laser device 32, an optical system 50 is disposed rather on the output portion side. In this embodiment, the laser beam is emitted in a parallel form from the light emitting portion 35 of the laser device 32, and a convex lens as the optical system 50 is disposed on the output portion side on an optical path of the parallel beam so that the laser beam is converged into a focus S near the skin surface. The distance from the skin contact portion 34 to the focus S of the laser beam is set, for example, in the range of 1 to 5 mm; in this embodiment, the distance is set to 3 mm.

The output portion 40 emits the laser beam with a wavelength having a vasodilating effect through the above-mentioned optical system. The wavelength of the laser beam having the vasodilating effect is in the range of 450 to 650 nm, and, in this embodiment, it is 650 nm, i.e., the laser beam is a red beam. In addition, the output of the output portion 40 is set in the range of 1 to 10 mW, and is 1 mW in this embodiment. Further, the output density of the laser beam is set in the range of 100 to 1550 mW/mm² for the above-mentioned reasons. While the irradiation with the laser beam is sustained irradiation in this embodiment, it may be pulsed irradiation.

Now, the operations of the light irradiating devices 1, 31 in the first and second embodiments of the present invention will be described below, based on the methods and results of experiments.

First, an experiment on a blood flow increasing action at a portion irradiated with a laser beam will be described.

A rat was used as an experimental animal. After the rat was anesthetized with pentobarbital, a probe (sensor portion diameter: 0.8 mm) of a blood flow meter (ADVANCE LASER FLOWMETER ALF21R, a product by ADVANCE Co., Ltd.) was put into secure contact with the inner side of the tip end of an auricle of the rat.

From the outer side of the auricle, a tip irradiation port of a probe (a stainless steel pipe with an outer diameter of 0.2 mm, fitted therein with a plastic fiber having a diameter of 0.125 mm) connected to a laser irradiating device (KTG LASERPRODUCT, a product by Kochi Toyonaka Giken Co., Ltd.) for irradiating with a laser beam having a wavelength of 532 nm was put on the direct upper side of the flow meter probe on the inner side of the tip end of the auricle, so as to clamp the auricle therebetween.

In addition to the probe composed of the stainless steel pipe having an outer diameter of 0.2 mm and fitted therein with the plastic fiber having a diameter of 0.125 mm, there was also prepared a probe composed of a stainless steel pipe having an outer diameter of 2 mm and fitted therein with a plastic fiber having a diameter of 0.6 mm. As the laser beam for irradiation therewith, LASERMATE-Q (a product by Coherent, Inc.) was used, and output measurement was carried out immediately before the experiment.

The value of blood flow in the auricle was measured by use of the above-mentioned blood flow meter, and the data was taken into a personal computer via a multiple recorder (NR500, a product by KEYENCE CORPORATION).

The output in irradiation with the laser beam was set in the range of 1 to 10 mW, and the irradiation time was set to be five minutes. The blood flow immediately before irradiation and the maximum blood flow during and after irradiation were read from the data, mean values of the blood flows were computed, and the blood flow increase ratio [(maximum blood flow after irradiation)/(blood flow immediately before irradiation); mean±SD] was computed. It is to be noted here that, since the blood flow increasing even after irradiation was shown in some cases, the maximum blood flow within a period of 10 minutes after the irradiation was read as a maximum action.

Temperature measurement was carried out by putting a temperature measuring probe instead of the blood flow meter probe into secure contact with the auricle, irradiating the auricle with the laser beam in the same manner as above, and continuously recording the temperature. The results are shown in Table 1 below.

TABLE 1

| Diameter of | Irradiation | Output density | Blood flow | | Increase | |
|---|---|---|---|---|---|---|
| fiber in probe | output | at output port | (ml/min/100 g) | | ratio | Number |
| (mm) | (mW) | (mW/mm²) | pre | post | (fold) | N |
| 0.125 | 3 | 250 | 21.4 ± 2.7 | 28.8 ± 3.3 | 1.34 | 5 |
| | 1 | 83 | 17.3 ± 1.2 | 20.2 ± 1.0 | 1.17 | 3 |
| 0.6 | 10 | 35 | 18.2 ± 2.1 | 26.6 ± 3.6 | 1.46 | 3 |
| | 3 | 11 | 19.7 ± 3.6 | 20.0 ± 2.7 | 1.02 | 3 |

As shown in Table 1, the laser beam radiated from the tip end of the fiber with a diameter of 0.125 mm onto the surface of the rat's auricle increased the dermal blood flow at the irradiated portion in an output-dependent manner. An output of 3 mW (output density: 250 mW/mm²) gave an increase in blood flow by 34%. When the skin temperature at the irradiated portion was measured, a temperature rise of about 4° C. was observed during the irradiation.

On the other hand, the laser beam radiated from the tip end of the fiber with a diameter of 0.6 mm had no effect on the dermal blood flow even at an output of 3 mW, but gave a blood flow increase of 46% at an output of 10 mW. In this case, the skin temperature was raised by about 2° C.

It is known that the dermal blood flow is increased by a rise in skin temperature, but the increase in blood flow is little when the temperature rise is about 2 to 4° C. Therefore, the blood flow increasing action under irradiation with a laser beam in this experiment is considered to be attributable mainly to the direct vasodilating action of the short-wavelength light. Incidentally, no influence was observed, to the naked eye, in the rat's auricular skin under any of the above-mentioned conditions.

Now, an experiment on a blood flow increasing action at a portion remote from the portion irradiated with a laser beam will be described below.

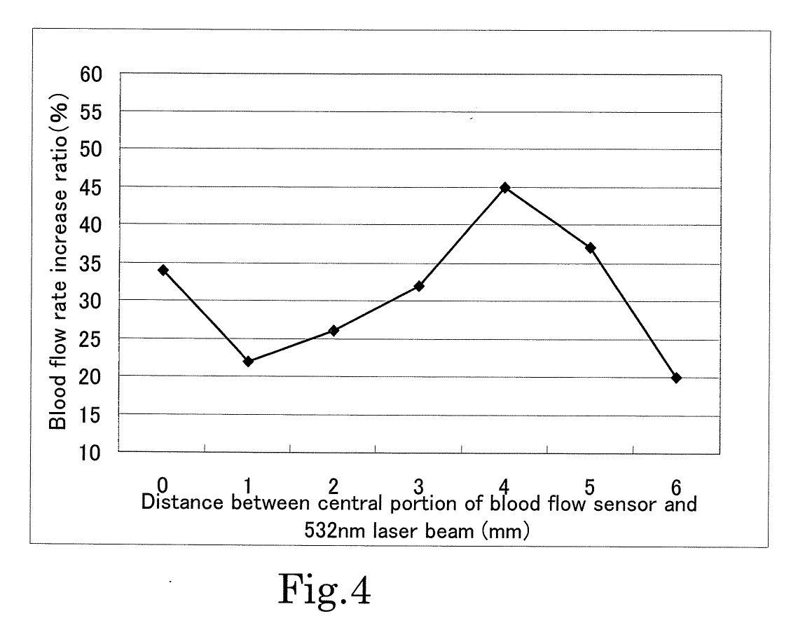

Blood flow measurement was conducted while moving a probe for irradiation with a laser beam from a position directly above the center of a sensor portion of a blood flow meter probe toward the base portion of the rat's auricle, by 1 mm at a time. The measurement results are shown in FIG. 4 (the number N of the measuring points was five).

As shown in FIG. 4, when the probe fitted therein with the 0.125 mm diameter fiber for irradiation with the laser beam was used, the blood flow increase ratio was halved upon merely moving the probe by 1 mm. However, when the probe was moved further toward the tip end of the auricle, the blood flow increase ratio was increased gradually, to reach an increase ratio of 450 when the probe was moved a distance of 4 mm. A further extension of the moving distance of the probe did not enlarge the blood flow increase ratio but rather reduced the increase ratio.

On the other hand, when the probe fitted therein with the 0.6 mm diameter fiber, only the blood flow at the irradiated portion was increased. Besides, when the probe was moved by 4 mm or above, no blood flow increase was observed at all.

This experiment revealed that the 532 nm laser beam radiated from the tip end of the very thin fiber with a diameter of 0.125 mm increased the dermal blood flow in a wide area, even at a low output, as contrasted to the irradiation with the laser beam emitted from the 0.6 mm diameter fiber. The blood flow increasing action having a peak at a distance of 4 mm from the irradiated portion was not recognized in the case of the 0.6 mm diameter fiber. Taking this into account, it is difficult to regard the blood flow increasing action as a direct action of light. Probably, the blood flow increasing action is an indirect action of the laser beam, due to stimulation of a dermal nerve.

In general, when a skin is given a strong acupuncture stimulus (by deep piercing with a thick acupuncture needle, or by burning moxa directly on the skin), the triple response appears on the skin, with the stimulated portion as a center. The triple response include a first reaction including rubor due to vascular dilation localized at the stimulated portion, a second reaction including wheal centered on the rubor, and a third reaction including a fugitive (returning to an original state reversibly and in a short time) flare with a diameter of several millimeters. Of these reactions, the first rubor and the wheal are considered to be inflammation reactions caused by chemical substances produced locally. On the other hand, the flare is considered to be a process in which, upon excitation of a certain kind of nociceptor (a polymodal receptor showing a reaction with a mechanical stimulus, a thermal stimulus or a chemical substance), the excitation conducted through a nerve is propagated reversely to the receptor (axon reflex), and chemical substances (substance P, calcitonin gene related peptide) librated from the receptor cause a local vascular dilation.

The output density of the laser beam emitted from the 0.125 mm diameter fiber used in the above experiments is higher than that of a low reaction level laser according to the related art. In this case, however, the total output is low (3 mW), and the laser beam diameter at the skin contact surface is smaller than the diameter of the thinnest needle (needle No. 1 having a diameter of 0.16 mm) used in acupuncture. Inflammation reactions such as rubor and wheal were not observed at all. Besides, not any reaction corresponding to flare was observed to the naked eye. However, the dermal blood flow was increased by 45% fugitively. Taking this into account, it is high possible that a reaction corresponding to the reversible flare occurred inside the skin. It is considered that the laser beam emitted from the 0.125 mm fiber caused a weak thermal stimulus locally in the skin, whereby the polymodal receptor was excited, and the excitation is conducted through axon reflex so as to dilate the blood vessels in the skin spaced from the light-irradiated portion. In short, it is considered that the very thin laser beam with a wavelength of 532 nm causes the vascular dilation directly in the irradiated portion, and causes the vascular dilation indirectly through axon reflex in the surroundings of the irradiated portion.

As has been described above, according to the light irradiating devices 1, 31 according to the first and second embodiments of the present invention, the laser beam with a wavelength having a vasodilating effect is emitted from each of the output portions 10, 40 at the tip end of the probe, and the output density is in the range of 100 to 1550 mW/mm². Therefore, the lesion portion, or a skin deep portion, can be irradiated with light having a high-vasodilating-effect wavelength at a low output energy. Thus, the light effect is useful for promotion of treatment of pains or wounds, such as postoperative or posttraumatic wound pain, posttraumatic pain, sore, bedsore, etc. and treatment of a wide range of diseases attended by circulatory incompetency such as arterioscelerotic blood vessel obstruction, diabetic circulatory incompetency, Raynaud's disease, Buerger's disease, oversensitiveness to cold, etc.

In addition, since a very thin laser beam is used, the laser beam can substitute for acupuncture needles, whereby effective spots for applying moxa or acupuncture can be stimulated.

Furthermore, the dilation of the dermal blood vessels has an effect of promoting absorption of drug through the skin, and, therefore, use of the light irradiating device according to the present invention in combination with a liniment or a patch type drug promises a promptness of the treatment.

### INDUSTRIAL APPLICABILITY

The light irradiating device according to the present invention is widely applicable as phototherapy apparatus. For example, with the light irradiating device, a lesion portion or a skin deep portion can be irradiated with light having a high-vasodilating-effect wavelength at a low output energy, which is useful for promotion of treatment of pains and wounds and for treatment of a wide range of diseases attended by circulatory incompetency. Besides, the light irradiating device can be a substitute for acupuncture needles.

1. A light irradiating device for emitting light including a wavelength having a vasodilating effect, from an output portion provided at the tip end of a probe,
wherein the output density of said light is 100 to 1550 mW/mm².

2. The light irradiating device as set forth in claim 1,
wherein a skin contact surface is formed at the tip end of said probe, said output portion is disposed at said skin contact surface; and
a laser beam at a wavelength having a vasodilating effect is emitted from said output portion, and the laser beam diameter at said output portion is 0.5 to 0.02 mm.

3. The light irradiating device as set forth in claim 2, wherein the wavelength of said laser beam having said vasodilating effect is 450 to 650 nm.

4. The light irradiating device as set forth in claim 2, wherein one said output portion or a plurality of said output portions are disposed at said skin contact surface.

5. The light irradiating device as set forth in claim 4, wherein the output of each said output portion is not more than 10 mW.

6. The light irradiating device as set forth in claim 2, wherein said output portion is opened in a projected portion rising from said skin contact surface of said probe.

7. The light irradiating device as set forth in any one of claim 2, wherein a touch sensor is provided in the periphery of said output portion, and said laser beam is emitted from said output portion only when said touch sensor is in operation.

8. The light irradiating device as set forth in claim 1,
wherein a skin contact portion is formed at the tip end of said probe, and said output portion is opened in said skin contact portion;
a laser beam at a wavelength having said vasodilating effect is emitted from said output portion, and the output of said output portion is 1 to 10 mW; and
an optical system is disposed between said output portion and a laser device, said optical system being so set that said laser beam is focused on a point in the vicinity of a skin surface.

9. The light irradiating device as set forth in claim 8, wherein the output density of said laser beam at the focus in the vicinity of said skin surface is 100 to 1550 mW/mm².

10. A light irradiating device for emitting light from an output portion provided at the tip end of a probe,
wherein the output density of said light is 100 to 1550 mW/mm², and the beam diameter of said light at said output portion is 0.5 to 0.02 mm.
